(19) United States
(12) Patent Application Publication
(10) Pub. No.: US 2018/0071177 A1
(43) Pub. Date: Mar. 15, 2018

(54) CHROMIC MICROCAPSULE COMPRISING CORE SEED AND PRESSURE SENSITIVE DESTRUCTIBLE WALL LAYER, AND PREPARATION METHOD THEREFOR

(71) Applicant: KPT LTD., Chungcheongbuk-do (KR)

(72) Inventors: Jae Uk LEE, Daejeon (KR); Byung Ho PARK, Cheongju-si, Chungcheongbukdo (KR); Ik Joo LEE, Ansan-si, Gyeonggi-do (KR); Yanfu JIANG, Yongin-si, Gyeonggi-do (KR); Woon Jang LEE, Cheongju-si, Chungcheongbuk-do (KR); Hailan JIN, Cheongju-si, Chungcheongbuk-do (KR)

(73) Assignee: KPT LTD., Chungcheongbuk-do (KR)

(21) Appl. No.: 15/519,754

(22) PCT Filed: Nov. 28, 2013
(86) PCT No.: PCT/KR2013/010906

§ 371 Date: Sep. 19, 2017

(30) Foreign Application Priority Data
Nov. 30, 2012 (KR) 10-2012-0137874

(51) Int. Cl. A61K 8/11; A61K 8/34; A61K 8/29; A61Q 1/02; C09B 67/02; C09B 67/08; B01J 13/22

(52) U.S. Cl. CPC A61K 8/11; A61K 8/345; A61K 8/29; A61Q 1/02; A61K 2800/63; C09B 67/0008; B01J 13/22; A61K 2800/43; C09B 67/0097

(57) ABSTRACT

The present invention provides a color-changing microcapsule composed of a core comprising an inner color layer having a color, a pressure-breakable wall layer surrounding the core, an optional outer color layer and an optional outermost layer, characterized in that said core comprises a core-seed and at least one inner color layer comprising a colorant and a binder, and said pressure-breakable wall layer comprises titanium dioxide particles and a binder.

Said color-changing microcapsule has a high durability during storage and handling and a high masking ability of inner color, can be easily ruptured by pressing, rubbing, wiping and/or scrubbing with hand or an implement such as cloths, sponge or paper to reveal or develop the color on the inner color layer as well as can maintain the stability for a long time when incorporated into a carrier of cosmetic formulations.

### TECHNICAL FIELD

The present invention relates to a color-changing microcapsule with core-seed and a pressure-breakable wall layer, preparation and use thereof, more specifically, to a color-changing microcapsule with core-seed structure comprising a core having a core-seed and at least one inner color layer, a pressure-breakable wall layer, an optional outer color layer and an optional outmost protective layer, preparation and use thereof.

### BACKGROUND ART

Use of microencapsulation is known in various fields. Microencapsulation involves the capturing of active ingredients within a shell which can be broken or dissolved, depending on the environment in which the active ingredient is to be released. Generally, however, microencapsulation has been utilized in the pharmaceutical and quasi-pharmaceutical field, to gradually release and maintain medication, vitamins or minerals by encapsulating the active ingredient within a shell which dissolves over time in the stomach.

The use of encapsulated materials to control release and improve the stability of composition is well established. Encapsulation efficiency can be improved by reducing the relative percentage of the protective wall material and increasing the quantity of the core encapsulate. Emphasis has been place on maximizing the absolute delivery of the encapsulated core material.

In recent, color-changing microcapsules have been proposed in cosmetic field. Said color changing microcapsules comprising a colorant, hide or do not show the colorant's color when they are not used, but will be ruptured to reveal or develop the color of the colorant when they are used or applied onto skin.

Korean Patent Laid-Open No. 10-2007-63908 discloses a friable capsule in which pigments are surrounded with a pressure-friable capsule membrane, said capsule membrane made from collagen, gelatin, agar or algin can be ruptured under pressure when used by a user to develop the pigment's color. However, said capsules have problems that they should be stored in a liquid matrix such as cosmetic carrier and the membrane is too friable under normal storage conditions as well as the colorant bleeds out through the capsule into the liquid matrix.

In addition U.S. Pat. No. 6,932,984 describes a method of preparing a microcapsule comprising 1) a step for dissolving or dispersing a colorant and at least one polymeric wall-forming materials selected from polyacrylate, polymethacrylate, cellulose ether, cellulose ester, polystyrene maleic anhydride copolymer in an organic solvent partially miscible with water, 2) a step for preparing an aqueous phase comprising an emulsifying agent, 3) a step for introducing the organic dispersing phase obtained in step 1) into the aqueous phase obtained in step 2) under gentle stirring to form an emulsion, 4) a step for extraction the organic solvent from the emulsion by adding an excess of water into said emulsion to obtain microcapsules, and 5) a step for separating, washing with water and drying the obtained microcapsules, or a step for introducing into about 5% alcohol solution and then separating and drying the obtained microcapsule. The present method for cosmetic raw materials containing microcapsules prepared by the patented process is now sold in the market in the trade name of Yellowcap, Redcap and Blackcap, respectively.

WO 2009/138978 discloses color-changing microcapsules containing a polymer-inorganic material shell or the polymer-plasticizer shell, wherein said inorganic material is selected from titanium dioxide, boron nitride, magnesium silicate, potassium, sodium magnesium hydroalumosilicate and/or magnesium myristate. The plasticizer is selected from tricaprylin, triaurine, tripalmitin mirastate and/or paraffin oil. However, the above-mentioned microcapsules are prepared by the emulsion method and have a diameter of only 70 μm.

On the other hand. EP 2 277 982 A discloses a color changing cleaning composition having a size range of 1-1000 μm and produced by a fluidized bed process, comprising a core (A) comprising a colorant and a shell (B) containing a wall forming polymer, a white pigment, such as titanium dioxide, barium sulfate or zinc oxide. The shell (B) described above is designed to dissolve fine pigment particles in water during and after hand rubbing process, and only after a predetermined time, for example, 2 to 4 minutes. That is, the aforementioned shell (B) cannot be regarded as a decompression-destructive wall because the decompression-destructive wall must be able to break down within a short time from the hand fineness, for example within 1 to 30 seconds. However, with some colorant-containing microcapsules it may be difficult to permanently retain the colorant over long periods of time and when subjected to different environments and conditions. This is true of pigments, oil soluble dyes, and water soluble dyes. Thus, some microcapsules described in the prior patents and publications have been found to gradually release the colorant, or to “bleed”, over a period of time when tested for prolonged periods at elevated temperatures. Color bleed occurs when a dye or pigment upon contacting with moisture and/or other ingredients in a formulation, migrates through or off of microspheres/microcapsules and it is more often when the shell including the pressure breakable wall surrounding the pigment is thin. Furthermore, some pigment-containing microcapsules are too fragile and are immediately broken down at the time of application so, while there is the fun of a sudden color change, it has not been possible to realize intermediate stages in this color change or to adjust the color gradation.

### PRIOR ART LITERATURE

### Patent Literature

(Patent Literature 1) Korean Published Patent Publication No. 10-2007-63908

(Patent Literature 2) U.S. Pat. No. 6,932,984

(Patent Literature 3) WO 2009/138978

(Patent Literature 4) EP 2 277 982 A

### DESCRIPTION OF THE INVENTION

### Technical Problem

One of the technical problem was to propose a stable color-changing microcapsule able to keep their properties over time, notably in term of coloring effect. Furthermore, some pigment-containing microcapsules may have some stability depending on the cosmetic composition with associated solvents/ingredients but several other pigment containing microcapsules cannot block the internal color completely to show unattractive gray color appearance.

Another technical problem was to propose microcapsules that will not break neither during the storage by absorbing water or moisture during storage nor during the shaking process of the composition containing the microcapsules before the use. An underlying technical problem was to propose the color-changing microcapsules able to survive in an extreme storage conditions (for instance at 45° C. for 3 months).

Another technical problem was to propose a color-changing microcapsule able to break easily and homogeneously when scrubbed onto the keratin material, giving a uniform color effect without residues or unpleasant colored dots.

At last, some microcapsules may give a discomfort and/or unfavorable feeling.

Thus there is a need to provide color-changing microcapsules able to solve at least one of the above-cited problem, and notably having improved color bleed resistance. In this respect, there is a need of colorant-containing microcapsules, which capsules retain good shatter resistance and exhibit improved bleed resistance.

There is also a need to provide color-changing microcapsules which allows the preferred coloration or gradation pattern to be adjusted by varying the method or intensity of application onto the skin or the use of microcapsules containing different colorants.

Thus there is a need for color-changing microcapsules containing pigments that do not provoke to the user a discomfort feeling when applied.

### Technical Solution

The present inventors have found that a color-changing microcapsule having a core-seed, an inner color layer surrounding the core-seed, and a pressure breakable titanium dioxide surrounding the inner color layer has a high durability during storage and handling, a high masking ability of inner color, and can be easily ruptured by pressing, rubbing, wiping and/or scrubbing with hand or an implement such as cloths, sponge or paper to reveal or develop the color on the inner color layer as well as can maintain the stability for a long time when incorporated into a carrier of cosmetic, etc., thus can solve at least one problem mentioned as above.

The present inventors also have found that, by additionally coating an outer color layer and/or an outermost shell onto said color-changing microcapsules, it is possible to obtain a color-changing microcapsule having a further improved durability during storage and handling as well as a long period stability even in a carrier.

### Effects of the Invention

The color-changing microcapsules according to the present invention have a high durability during storage and handling, high masking ability of inner color, can be easily ruptured by pressing, rubbing, wiping and/or scrubbing with hand or an implement such as cloths, sponge or paper to reveal or develop the color on the inner color layer as well as can maintain a long period stability even in cosmetic carriers.

### BRIEF DESCRIPTION OF DRAWINGS

FIG. 1 is a schematic diagram illustrating a structure of color-changing microcapsule of the present invention.

FIGS. 2 to 10 represent a schematic diagram showing the core-shell structure of color-changing microcapsules prepared according to Examples 6-14, respectively.

### MODE OF INVENTION

The first object of the present invention is to provide a core-seed (A) color-change microcapsule (B) composed of a core comprising an inner color layer having a color, a pressure-breakable wall layer (C) surrounding the core, an optional outer color layer (D) and an optional outermost protective layer (E), wherein said core comprises a core-seed and at least one inner color layer comprising a colorant and a binder, and said pressure-breakable wall layer comprises titanium dioxide particles and a binder.

Specifically, the present invention provides a color-changing microcapsule having an average amount between 50-1500 μm and having a core-shell structure, wherein said the core comprises a core seed (A) and one or more inner color layers (B), wherein said shell comprises a pressure breakable wall layer (C):

(A) A core-seed having an average of 500 nm-150 μm and containing no colorant: and having sugar alcohol:

(B) At least having one or more inner color layer:
One or more colorants, and
A binder comprising at least one wall forming material and at least one lipid-based material and

(C) A pressure breakable wall layer having a thickness of 10 to 500 μm and comprising:
Titanium dioxide particles, and
A binder comprising at least one wall forming material and at least one lipid-based material.

In the present invention, the core of the color-change microcapsule may comprise two or three inner color layers as follows.

(B-1) First inner color layer comprising:
At least one colorant, and
A binder comprising a wall-forming material and at least one lipid-based material; and

(B-2) Second inner color layer comprising:
At least one colorant, and
A binder comprising a wall-forming material and at least one lipid-based material; and

(B-3) Third inner color layer comprising:
At least one colorant, and
A binder comprising a wall-forming material and at least one lipid-based material; and

Here, the above-mentioned colorants, wall-forming materials and lipid-based materials used in (B-1), (B-2) and (B-3) are the same or different from each other.

In another preferred embodiment according to the invention, the shell of the color-change microcapsule may comprise one or both of the following (D) and (E):

(D) At least one or more outer color layer surrounding the pressure breakable wall layer and comprising:
At least one colorant, and
A binder comprising a wall-forming material and at least one lipid-based material: and

(E) An outermost protective layer surrounding the pressure breakable wall layer or outer color layer and comprising:

A shell-forming polymer selected from the group consisting of shell rock, polyacrylates, polymethacrylates, cellulose ethers, cellulose esters, polystyrene maleic anhydride copolymers and mixtures thereof.

A second object of the present invention is to provide a process for producing color-changing microcapsules comprising a pressure-breakable wall layer comprising the following steps:

(a) A core seed (A) particles are being prepared,

(b) The core seed particles are coated with a solution in which a colorant and a binder are dispersed or dissolved to form an inner color layer (B);

(c) The particles obtained in the step (b) are coated with a solution in which titanium dioxide particles and binder are dispersed or dissolved to form a pressure breakable wall layer (C);

wherein the binder described above comprises a wall-forming material and a lipid based material, wherein the wall-forming material and the lipid-based material described above are the same or different from each other.

In one preferred embodiment of the present invention, the above-mentioned step (b) comprises the following steps (b-1) and (b-2):

(b-1) core-seed (A) particles are coated with a solution in which a colorant and a binder are dispersed or dissolved to form a first inner color layer (B-1),

(b-2) The particles obtained in the step (b-1) are coated with a solution dispersed or dissolved in the same or different colorant and binder as those used to form a second inner color layer (B-2):

wherein the binder described above comprises a wall-forming material and a lipid-based material, wherein the wall-forming material and the lipid-based material described above are the same or different from each other.

In another preferred embodiment of the present invention of a process for producing color-changing microcapsules comprises one or both of steps (d) and (e):

(d) Coating the particles obtained in the step (c) with a solution dispersed or dissolved in the same or different colorants and binders as those used in the step (b-1) or (b-2),

(e) Coating the particles obtained in the step (c) or (d) with a solution in which a shell-forming polymer is dispersed or dissolved to form an outermost protective layer (E):

In one preferred embodiment according to the present invention, each step (b), (b-1), (b-2), (c), (d) and/or (e) is carried out by a fluidized bed process or a fluidized bed coating process.

In a preferred embodiment of the present invention, the solution used in this step may be water or a low boiling organic solvent such as methylene chloride, methanol or ethanol as the solvent.

Presented below, the present invention is explained in details with reference to drawings.

In the present invention, the color-changing microcapsule having a core comprising a colorant, and a shell comprising a pressure-breakable breakable wall layer, comprises a core having at least one inner color layer, a pressure-breakable wall layer surrounding the core, an optional outer color layer and an optional outmost shell layer.

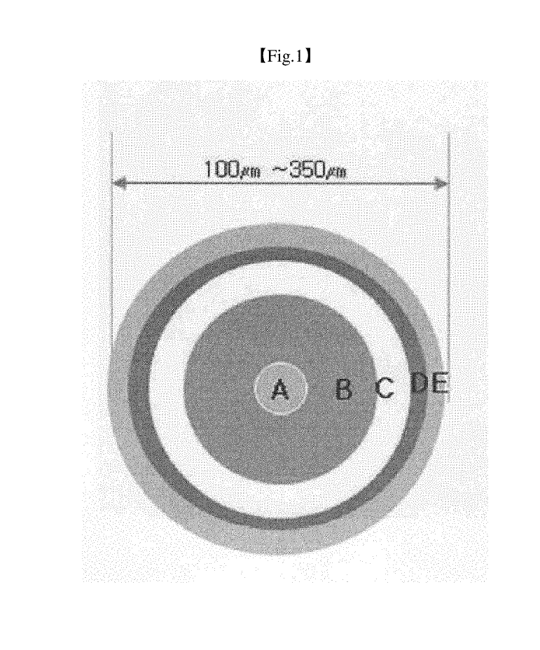

FIG. 1 is a schematic diagram illustrating the structure of a color-changing microcapsule according to the present invention, wherein A represent a core-seed, B represents an inner color layer, C represents a pressure-sensitive titanium dioxide particles layer, D represents an outer color layer, and D is an outmost protective layer.

Although the color-changing microcapsule illustrated in FIG. 1 has a particle size of 100-350 μm, the color-changing microcapsule according to the present invention a mean particle size is generally about 50 μm or more, specifically 70 μm or more, particularly 80 μm or more, preferably 90 μm or more, more preferably 100 μm or more, of and about 1500 μm or less, specifically 1200 μm or less, particularly 1000 μm or less, preferably 800 μm or less, more preferably 700 μm or less.

Alternatively, the color-changing microcapsule according to the present invention has a mean particle size of about 14-280 mesh (around 1400 μm-50 μm), particularly about 24-150 mesh (around 800 μm-100 μm).

1. Core-Seed

In the context of the present invention, the term “core-seed” or “core-seed particle” refers to a sort of a nuclei growth particle or a central basic particle used for the formation and growth of an inner color layer of the core, and it plays a role of a support on which the inner layer is coated by a fluidized bed coating process.

In the present invention, a core-seed does not have any color, that is, does not contain any colorant and is a singular or granular minute particle which has a solid or crystalline form at a room temperature. In the present invention, core-seed can be selected from inorganic or organic materials which have a high water-solubility or are water-soluble or water-dispersible, preferably selected from water-soluble or water-dispersible organic materials.

In a preferred embodiment, core-seed can be selected from a group consisting of sugars, salts and sugar alcohols, preferably selected from sugar alcohols which can be derived from mono- or di-saccharides, such as erythritol, threitol, arabitol, xylitol, ribitol, mannitol, sorbitol, galactitol, iditol, inositol, volemitol, or the like. In a particularly preferred embodiment, core-seed comprises mannitol, and more particularly, it consists of mannitol only.

In another embodiment, core-seed may comprise, as a hydrophilic polymer, a cellulose polymer (e.g. carboxymethylcellulose), a starch polymer (e.g. unmodified natural starch) and mixtures thereof.

The core-seed may be used in terms of total weight of core in an amount of 1-50% by weight, preferably 3-40% by weight, more preferably 5-30% by weight, and particularly 8-25% by weight.

Said sugar alcohol such as mannitol may be used in terms of total weight of core-seed in an amount of 1-100% by weight, preferably 2-100% by weight, more preferably 5-100% by weight, and particularly 100% by weight.

The shape of core-seed is not particularly limited but may have a prismatic, cylindrical or spherical shape or a shape similar to them.

The size of core-seed is not particularly limited and may be suitably chosen according to the finally desired color-changing microcapsule. For example, the average diameter of core-seed particles is generally about 500 nm or more, particularly 1 μm or more, preferably 5 μm or more, more preferably 10 μm or more, and about 150 μm or less, particularly 120 μm or less, preferably 100 μm or less, more preferably 80 μm or less.

2. Inner Color Layer

In the present invention, inner color layer can be formed by coating a core seed with a solution having a colorant and a binder, for example, by a fluidized bed coating process.

The core can comprise one or more inner color including, for example, first inner color layer, second inner color layer and third inner color layer, etc., wherein the colorants and binders contained in each inner color layers are the same or different from each other.

When the core comprises two or more inner color layers, a first inner color layer can be formed by coating a core-seed with a solution for first inner color layer comprising a first colorant and a first binder, a second inner color layer can be formed by coating the first inner color layer with a solution for second inner color layer comprising a second colorant and a second binder, and in the same manner, a third inner color layer, a fourth inner color layer and further inner color layer can be formed. Each coating process can be performed by a fluidized bed coating process. Each inner color layer can be circumferentially extended by centering the core-seed.

The binder can be used in an amount that colorant will not fall apart or separate from the layer during the coating process and/or after the removal of solvent, and generally can be used in an amount selected from 0.5 to 15% by weight, preferably 1-10% by weight, particularly 1.5-9% by weight, and more particularly 2-8% by weight in the terms of total weight of inner color layer.

The colorant is the main ingredient of inner color layer, and therefore, is used, in terms of total weight of inner color layer, in an amount of at least 40% by weight, preferably at least 75% by weight and more preferably at least 95% by weight of the inner color layer.

The inner color layer may be included in an amount of 50-99% by weight, preferably 97-60% by weight, particularly 70-95% by weight, and more particularly 75-93% by weight, based on the total weight of the core.

3. Pressure-Breakable Wall Layer or Titanium Dioxide Particle Layer

The color-changing microcapsule of the present invention has a pressure-breakable wall layer or pressure-breakable titanium dioxide particle layer, wherein the titanium dioxide particles are discontinuously dispersed in the layer and linked to each other by a binder.

In the context of the present invention, the term “pressure-breakable” or “pressure-friable” means that a rupture can be easily made by pressing, rubbing, wiping and/or scrubbing with hand or an implement such as cloths, sponge or paper.

In the present invention, a pressure-breakable titanium dioxide particles layer can comprise particles of titanium dioxide and a binder, and said binder can comprise a wall-forming material and a lipid based material.

In the pressure-breakable wall layer of the present invention, it is believed that the titanium dioxide particles lodged in the wall-forming materials will break the pressure-breakable wall layer in an irreversible manner and facilitate or increase the disintegration or dissolution of said wall layer. Further, it is also estimated that titanium dioxide particles do a critical role for the strength, the durability, the pressure-breakability, and the after-feeling of the wall layer.

The titanium dioxide particle layer, of which thickness can vary depending on the amount of titanium dioxide used and/or the type of binder, may have a thickness of usually 10 μm or more, preferably 20 μm or more, more preferably 30 μm or more, particularly 40 μm or more, commonly 500 μm or less, preferably 400 μm or less, more preferably 300 μm or less, particularly 200 μm or less.

Alternatively, the titanium dioxide particle layer can have a content of 5-70% by weight, preferably 10-60% by weight, more preferably 15-50% by weight, particularly 20-40% by weight in term of the total weight of microcapsule.

In the present invention, the mean diameter or size of titanium dioxide particles is not specifically limited but has a mean diameter of usually 10 nm-20 μm, preferably 50 nm-10 μm, more preferably 100 nm-5 μm, and particularly 150 nm-5 μm. The mean diameter or size of less than 10 nm of titanium dioxide particles may result to a decrease in the pressure-breakable ability, and the mean diameter of more than 20 μm may make difficult the formation of titanium dioxide particles layer. Titanium dioxide particles having a first particle size of less than the above range but having a second particle size falling down the above particle size range can be applicable in the present invention.

The content of titanium dioxide particles in the pressure-breakable wall layer can be selected from usually 5-99% by weight, preferably 10-95% by weight, more preferably 15-90% by weight, particularly 20-85% by weight, in terms of total weight of the pressure-breakable wall layer.

4. Outer Color Layer

The color-changing microcapsule additionally comprises an optional outer color layer onto the pressure-breakable titanium dioxide particles layer. The outer color layer can be formed by coating the titanium dioxide particles layer with a solution having a colorant and a binder, for example, by the fluidized bed process.

The colorant and binder used in the outer color layer can be the same or different from those used in the inner color layer.

In general, the outer color layer is given to impart a visual color different from white color issued from the titanium dioxide particle layer and/or the color of inner color layer. Therefore, a colorant in the outer color layer can be used in an amount that does not disturb the color developed by the inner color layer when the microcapsules are applied to skins.

The content of an outer color layer can be selected, in terms of the total weight of core, from 1-60% by weight, preferably 2-50% by weight, more preferably 3-40% by weight, particularly 4-30% by weight. However, the content of a colorant in the outer color layer may be selected, in terms of total weight of colorants in the inner color layer, from 0.01-5% by weight, preferably 0.05-4.5% by weight, more preferably 0.1-4% by weight, particularly 0.5-3.5% by weight.

The content of a colorant in an outer color layer may be additionally increased if the color of the outer color layer would not disturb the color of the inner color layer. A person skilled in the art can choose the color and content of a colorant in an outer color layer in an appropriate manner by considering the color and content of colorants contained in inner color layers and the desired color to be finally developed.

5. Outermost Protective Layer

Microcapsule of the present invention can comprise a protective outermost protective layer onto a pressure-breakable wall layer or an additional outer color layer to protect the microcapsule against moisture in the air during storage or to ensure a long period stability of the microcapsule in a cosmetic carrier such as water, alcohol, etc.

The outermost protective layer can be made from at least one selected from the group consisting of shellac, polyacrylate, polymethacrylate, cellulose ether, cellulose ester and polystyrene-maleic anhydride copolymer.

The content of said outermost protective layer is selected, in terms of total weight of microcapsule, from 0.1-20.0% by weight and preferably 0.5-15% by weight. When the content of the outmost shell is less than 0.1% by weight, the shell coating may be meaningless, and when it is more than 20.0% by weight, a feeling of foreign substances may be caused.

The thickness of the outermost protective layer is usually 5 μm, preferably 10 μm or more, more preferably 15 μm or more, especially 20 μm or more, usually at most 200 μm, preferably 150 μm or less, more preferably 120 μm or less, but it is not strictly limited.

6. Colorant or Coloring Agent

In the present invention, “colorant” include any synthetic or natural, or organic or inorganic pigments, dyes or lakes, and any colorants approved for use in cosmetics by CTFA and the FDA used in cosmetic formulations.

In the present invention, the colorant may be water-soluble or water-dispersible, or oil-soluble or oil-dispersible or with limited solubility in water.

In the present invention, thus the term “colorant” refers to organic pigments such as dyes selected from any of the well-known FD&C or D&C dyes, inorganic pigments such as metal oxides, or lakes such as the ones based on cochineal carmine, barium, strontium, calcium or aluminum and any combination (blend) thereof.

In the present invention, the following colorants can be mentioned:
carmin of cochenille;
organic pigments of azoïques, anthraquinoniques, indigoïdes, xantheniques, pyreniques, quinoliniques, de triphenylmethane, de fluorane colorants;
of acid colorants such as azoïques, anthraquinoniques, indigoïdes, xantheniques, pyreniques, quinoliniques, de triphenylmethane, de fluorane colorants, insoluble salts of sodium, potassium, calcium, baryum, aluminum, zirconium, strontium, titanium, these colorants may include at least one carboxylic or sulfonic acid group.

As to particular examples of organic pigments, those having the following trade names can be mentioned:
D&C Blue no 4, D&C Brown no 1, D&C Green no 5,
D&C Green no 6, D&C Orange no 4, D&C Orange no 5, D&C Orange no 10,
D&C Orange no 11, D&C Red no 6, D&C Red no 7, D&C Red no 17, D&C Red no 21, D&C Red no 22, D&C Red no 27, D&C Red no 28, D&C Red no 30, D&C Red no 31, D&C Red no 33, D&C Red no 34, D&C Red no 36, D&C Violet no 2, D&C Yellow no 7, D&C Yellow no 8, D&C Yellow no 10, D&C Yellow no 11, FD&C Blue no 1,
FD&C Green no 3, FD&C Red no 40, FD&C Yellow no 5, FD&C Yellow no 6.

In preferred embodiments, the colorant is an inorganic pigment, more preferably a metal oxide.

Advantageously, the colorants of the multi-layer microcapsules are primary metal oxides selected from iron oxides, titanium dioxide, aluminum oxide, zirconium oxides, cobalt oxides, cerium oxides, nickel oxides, tin oxide or zinc oxide, or composite oxides, more preferably an iron oxide selected from red iron oxide, yellow iron oxide or black iron oxide, or a mixture thereof.

A person skilled in the art knows how to choose colorants and combinations of colorants to produce a desired color effect or color change.

In preferred embodiments, if white is the desired color to be developed by the color-changing microcapsule, a white colorant such as titanium dioxide can be chosen as a colorant for inner color layer. In such case, the inner color layer may be substantially the same or similar to the titanium dioxide particles layer, and thus, it can be understood that a titanium dioxide particle layer can simultaneously plays both roles of an inner color layer and pressure-breakable wall layer.

Meanwhile, a color may be achieved from one colorant alone, but most colors can be generally achieved from mixed colorants by changing the composition of colorants. Therefore, in the context of the present invention, the term “a (the) colorant” may cover both of “one colorant” and “a mixture of colorants”, if there is no specific restriction.

In a preferred embodiment, core and pressure breakable wall layer can be manufactured at least in part as metal oxides, preferably an iron oxide for cores and titanium dioxide for pressure breakable walls.

7. Binder

In general, it is difficult to form a coating layer by using only colorant component or particles without using any binder. Further, even if a coating layer without a binder is formed with difficulty, such coating layer may be easily damaged or ruptured or any components or particles may be easily removed from the coating layer. Therefore, a binder is commonly employed in order to proceed the coating process and to improve the durability of coating layer.

In the present invention, the binder comprises both of a wall-forming polymer as a wall-forming material and a lipid-base material as coating base.

In general the coating base refers to a hydrophilic coating base, a hydrophobic coating base, or lipid-based coating base. Since the hydrophilic coating base may be extracted together with colorant into cosmetic carrier and the hydrophobic coating base may give a feeling of foreign substances due to its tow strong film property, it is preferable to employ a lipid-base coating base.

According to a particular embodiment of this invention, such lipid based material may have amphiphilic properties, that is to say having an apolar part and a polar part. Such lipid-based material can include at least one or several C₁₂-C₂₂ fatty acids chain such as selected from stearic acid, palmitic acid, oleic acid, linoleic acid, linolenic acid, etc and mixtures thereof. Preferably these fatty acids chains are hydrogenated. Eventually, these fatty acid chains may be the apolar part of a lipid-based material.

According to a particular embodiment of this invention, said lipid-based materials can be selected form the group consisting of a phospholipid such as phosphatidylcholine, phosphatidylethanolamine, phosphatidic acid or phosphatidylserine, a sphingolipid such as sphingosine-1-phosphate or sphingomyelin and ceramide, preferably ceramide or lecithin which is a phospholipid mixture, particularly hydrogenated lecithin.

One of the advantages of such lipid-based materials is that they can also act as wall-forming materials. Thus, in a particular variation of the present invention, the binder comprised with lipid-based materials alone does not depart from the scope of the present invention, with no or little use of a wall-forming polymer such as a hydrophilic polymer.

The amount of the lipid-based material to be used can be determined by considering the type and amount of the wall-forming material as well as other components such as colorant and/or titanium dioxide particles. However, in general, the content of the lipid-based material is in the range of 0.1-30% by weight, in particular 0.2-25% by weight, preferably 0.3-20% by weight, and more preferably, may be selected from 0.4-20% by weight. If the content of the lipid base material is less than 0.1% by weight, the breaking property or the dissolving ability may be lowered, and if it is more than 25.0% by weight, the durability may be decreased or the durability and stability during processing and storage may be deteriorated.

In the present invention, the wall-forming polymer is selected from hydrophilic polymers. The term “hydrophilic polymers” means a polymer which can form hydrogen bond with water or alcohol compounds (especially elected from lower alcohols, glycol and polyol), particularly those having O—H, N—H and S—H bonds in the molecule.

Said hydrophilic polymer can be selected from the following polymers or mixture thereof:
acrylic or methacrylic acid homopolymers or copolymers or salts and esters thereof and in particular the products sold under the names Versicol F or Versicol K by the company Allied Colloid, Ultrahold 8 by the company Ciba-Geigy, and polyacrylic acids of Synthalen K type, and salts of polyacrylic acids, especially sodium salts, (corresponding to the INCI name sodium acrylate copolymer) and more particularly a crosslinked sodium polyacrylate (corresponding to the INCI name sodium acrylate copolymer (and) caprylic/capric triglycerides) (sold under the name Luvigel EM by the company);
copolymers of acrylic acid and of acrylamide (sold in the form of the sodium salt thereof under the names Reten by the company Hercules), the sodium polymethacrylate (sold under the name Darvan No. 7 by the company Vanderbilt), and the sodium salts of polyhydroxycarboxylic acids (sold under the name Hydagen F by the company Henkel);
polyacrylic acid/alkyl acrylate copolymers, preferably modified or unmodified carboxyvinyl polymers; the copolymers most particularly preferred according to the present invention are acrylate/C₁₀-C₃₀-alkylacrylate copolymers (INCI name: Acrylates/C₁₀₋₃₀ Alkylacrylate Cross polymer) such as the products sold by the company Lubrizol under the tradenames Pemulen TR1, Pemulen TR2, Carbopol 1382 and Carbopol ETD2020, and even more preferentially Pemulen TR-2;
alkylacrylic/alkylmethacrylic acid copolymers and their derivatives notably their salts and their esters, such as the copolymer of ethyl acrylate, methyl methacrylate and low content of methacrylic acid ester with quaternary ammonium groups (provided under the tradename of EUDRAGIT RSPO from Evonik Degussa);
AMPS (polyacrylamidomethylpropanesulfonic acid partially neutralized with aqueous ammonia and highly crosslinked) (sold by the company Clariant);
AMPS/acrylamide copolymers such as the products Sepigel or Simulgel sold by the company SEPPIC, especially a copolymer of INCI name Polyacrylamide (and) C13-14 Isoparaffin (and) Laureth-7;
polyoxyethylenated AMPS/alkyl methacrylate copolymers (crosslinked or non-crosslinked) of the type such as Aristoflex HMS sold by the company Clariant;
anionic, cationic, amphoteric or nonionic chitin or chitosan polymers;
cellulose polymers and derivatives, preferably other than alkylcellulose, chosen from hydroxyethylcellulose, hydroxypropylcellulose, hydroxymethylcellulose, ethylhydroxyethylcellulose and carboxymethylcellulose, and also quaternized cellulose derivatives; in a preferred embodiment, the cellulose polymers is a carboxymethylcellulose;
Starch polymers and derivatives, eventually modified; in a preferred embodiment, the starch polymer is a natural starch;
vinyl polymers, for instance polyvinylpyrrolidones, copolymers of methyl vinyl ether and of malic anhydride, the copolymer of vinyl acetate and of crotonic acid, copolymers of vinylpyrrolidone and of vinyl acetate; copolymers of vinylpyrrolidone and of caprolactam; polyvinyl alcohol;
optionally modified polymers of natural origin, such as galactomannans and derivatives thereof, such as konjac gum, gellan gum, locust bean gum, fenugreek gum, karaya gum, gum tragacanth, gum arabic, acacia gum, guar gum, hydroxypropyl guar, hydroxypropyl guar modified with sodium methylcarboxylate groups (Jaguar XC97-1, Rhodia), hydroxypropyltrimethylammonium guar chloride, and xanthan derivatives;
alginates and carrageenans;
glycoaminoglycans, hyaluronic acid and derivatives thereof;
mucopolysaccharides such as hyaluronic acid and chondroitin sulfates, and mixtures thereof. Preferably, the hydrophilic polymers according to the present invention can be selected from the group consisting of polysaccharides and its derivatives, homopolymers or copolymers of acrylic or methacrylic acid or salts and esters thereof, and their mixture. Said polysaccharides and derivatives can be selected from chitosan polymers, chitin polymers, cellulose polymers, starch polymers, galactomannans, alginates, carrageenans, mucopolysaccharides, and their derivatives, and the mixture thereof.

In one preferred embodiment, the hydrophilic polymers can be selected from the group consisting of corn starch, (meth)acrylate or (alkyl)(meth)acrylate and its salts or copolymer of ester derivatives, particularly polymethyl methacrylate, cellulose or its derivatives such as carboxymethylcellulose (CMC), cellulose ester and ether and aminocellulose, and mixture thereof.

Preferred homo- and/or co-polymer of methacrylic acid and/or methacrylic acid ester are those wherein the copolymer of methyl methacrylate and ethyl acrylate has a molecule weight from 750 to 850 kDa.

The hydrophilic polymer(s) used as a wall-forming material in the present invention are not cross-linked.

The amount of polymer or wall-forming polymer used can be determined by considering the type and amount of the colorant, the titanium dioxide particles and/or the lipid-based material. In general, the content of the polymer or wall-forming polymer is in the range of 0.1 to 30% by weight, in particular 0.2 to 25% by weight, preferably 0.3 to 20% by weight, and more preferably, can be selected from 0.4 to 20% by weight

8. Color-Changing Microcapsules

In the present invention, the term “microcapsule”, as used herein, refers to a substantially spherical microcapsule containing at least one layered coating entrapping at least one colorant and surrounding a core chemically different from the coating.

The term “multi-layer microcapsule” refers to a microcapsule consisting of an inner core surrounded by a coating based on one or more inner layer(s) and one outer layer. The one or more inner layer(s) forming the multi-layer coating of the multi-layer microcapsule and the single layer of the outer core microcapsule may be formed of the same or different wall-forming organic compound(s).

According to the present invention, the term “color-changing microcapsule” or “color-changing beads” means a microcapsule or bead wherein the color before application is different from the color after application, this difference being visible to the naked eyes. In the present invention, a pressure-friable or pressure-breakable wall layer is provided, which can be easily ruptured by pressing, rubbing, wiping and/or scrubbing with hand or an implement such as cloths, sponge or paper.

According to the present invention, at least 60%, in particular at least 70%, preferably at least 80%, and more preferably at least 90% of the color-changing microcapsule particles can be made within 1 minute, especially within 1 to 40 seconds, preferably within 1 to 30 seconds, more preferably within 1 to 20 seconds, of the coloring agents of the core after being polished and/or rubbed. However, it is needless to say that the breaking time can be changed according to the necessity, depending on the thickness of the protective layer and/or the pressure breakable wall layer and the installation of the additional wall.

9. Fluidized-Bed Coating Process

In a preferred embodiment, the microcapsules can be produced by a fluid bed process or a similar process. While the granulation by the spray drying method induces matrix particles with granular particles by particle agglomeration or randomly dispersed core material in the polymer medium, the specificity of the fluidized bed process is to concentrate one core or one or more outer layers concentrically, then it is possible to derive an actual capsule having a core shell structure enclosed therein.

Fluid bed process is disclosed by example in ‘Fluid-Bed Coating, Teunou, E.; Poncelet, 2005, D. Food Science and Technology (BocaRaton, Fla., United States), Volume 146 Issue Encapsulated and Powdered Foods, Pages 197-212.

A man skilled in the art knows how to adjust air quantity, liquid quantity and temperature allowing reproducing a microcapsule according to the invention.

Preferably a fluid bed process implemented includes Würster process and/or tangential spray process. Such process allows, contrary to pelletizing process, to conduct to spherical capsules with core surrounded by one or more circumferential layers.

In the present invention, by combining three or more compounds (ex: sugar alcohols, wall forming material, lipid-based material) in the microcapsule of different hardness and/or water solubility, it is possible to adjust the time required for colorant-encapsulated microcapsules to break down on the skin so that, by varying the method or intensity of application onto the skin, it is possible to adjust the preferred coloration or gradation pattern.

Thus, according to a preferred embodiment, the multi-layers coating contains at least starch as polymer with at least one lipid-based material and preferably lecithin.

According to an advantageous embodiment, the microcapsules according to the invention include at least one monosaccharide or its derivatives and at least one polysaccharide or its derivatives. According to a preferred embodiment, the microcapsules include a core comprising a monosaccharide polyols preferably chosen from mannitol, erythritol, xylitol, sorbitol and a polysaccharides including ose (at least D-glucose unit).

According to a preferred embodiment, the microcapsules include three or more colorants in different layers.

According to a preferred embodiment, the microcapsules additionally includes lipid-based material chosen from phospholipids, advantageously selected from phosphoacylglycerol and in particular lecithin.

In a particular embodiment, the core contains mannitol, starch polymer and cellulose derivatives and optionally lipid base material. In such a case, the starch polymer is the main ingredient, i.e. the weight amount of starch is greater than the respective amount of mannitol, cellulose derivative and lipid based material of the core.

As examples of color-changing microcapsules according to the invention, we may refer to the following microcapsules comprising following ingredients:
Pink spherical microcapsule: containing titanium dioxide, mannitol, hydrogenated lecithin, synthetic fluorphlogopite, red 30 lake, corn starch, tin oxide, having 60-200 Mesh particle size;
Ash gray spherical microcapsule: containing mannitol, iron oxide red, iron oxide yellow, iron oxide black, hydrogenated lecithin, titanium dioxide, corn starch, having 60-200 Mesh particle size.

In the present invention, an organic solvent may be employed in the preparation of coating solution used in the fluidized bed coating process. The organic solvent which can be used in the present invention is not specifically restricted but preferably includes methylene chloride, methanol, ethanol, and mixture thereof. It is possible to employ any organic solvent if it can dissolve or disperse the polymers and/or lipid-based materials, has a boiling point less than that water, and has a low residual toxicity.

The present invention will be further explained by the examples, but is not restricted by them. Unless otherwise specified in the examples, percentage and ratio are based on weight, lecithin means hydrogenated lecithin, and the name of the substance or substance is given when the name of the contained substance or substance is clear.

### Example 1: Preparation of a Core-Shell Capsule Having Inner Brown Color and Outer White Color

Mannitol (spray dried mannitol: Pearitol 100SD) is used as core-seed.

To a mixed solution of 1600.0 g of methylene chloride and 1600.0 g of ethanol, 120.0 g of ceramide (Ceramide PC104) and 120.0 g of hydrogenated lecithin (Lipoid S 100-3) are added and completely dissolved at around 40□. To the resulting reaction mixture, 1260.0 g of iron oxide yellow, 252.0 g of iron oxide red and 45.36 g of iron oxide black are added and well dispersed with a homogenizer to prepare an inner color coating solution.

347.70 g of Mannitol is introduced into a fluidized bed coating system (Glatt GPOG 1, bottom spray) and subjected with a coating at the condition of 500 me/h of feeding rate of the inner color coating solution to obtain core particles having a core seed coated with an inner color layer.

Thereafter, to a mixed solution of 720.0 g of methylene chloride and 720.0 g of ethanol, 36.0 g of ceramide and 36.0 g of hydrogenated lecithin are added and dissolved at around 40° C. To the resulting reaction mixture, 600.0 g of particular titanium dioxide (TiO₂) is added and well dispersed with a homogenizer to prepare a titanium dioxide particle coating solution. A coating with the resulting titanium dioxide particle coating solution is proceeded by a fluidized bed process to obtain particles having an inner color layer coated with a titanium dioxide particle layer.

Then, 300.0 g of shellac is dissolved in 3000 g of ethanol to prepare an outermost protective layer coating solution. A coating onto the above titanium dioxide particle layer is proceed by a fluidized bed process to obtain a color-changing microcapsule having a titanium dioxide particle layer coated with an outermost protective layer.

### Example 2: Preparation of a Core-Shell Capsule Having Inner Yellow Color and Outer White Color

A core-shell capsule is prepared in the same manner as in Example 1 except for using 1557.36 g of iron oxide yellow instead of the mixed colorants consisting of 1260.0 g of iron oxide yellow, 252.0 g of iron oxide red and 45.36 g of iron oxide black as the inner color in the preparation of the inner color coating solution.

### Example 3: Preparation of a Core-Shell Capsule Having Inner Red Color and Outer White Color

A core-shell capsule is prepared in the same manner as in Example 1 except for using 1557.36 g of iron oxide red instead of the mixed colorants consisting of 1260.0 g of iron oxide yellow, 252.0 g of iron oxide red and 45.36 g of iron oxide black as the inner color in the preparation of the inner color coating solution.

### Example 4: Preparation of a Core-Shell Capsule Having Inner Black Color and Outer White Color

A core-shell capsule is prepared in the same manner as in Example 1 except for using 1557.36 g of iron oxide black instead of the mixed colorants consisting of 1260.0 g of iron oxide yellow, 252.0 g of iron oxide red and 45.36 g of iron oxide black as the inner color in the preparation of the inner color coating solution.

### Example 5: Preparation of a Core-Shell Capsule Having Inner Red Color and Outer Green Color

The same procedure as in Example 1 is repeated to the step for forming a titanium dioxide particles layer.

Thereafter, to a mixed solution of 400.0 g of methylene chloride and 400.0 g of ethanol, 20.0 g of ceramide and 20.0 g of hydrogenated lecithin are added and dissolved at around 40□. To the resulting reaction mixture, 40.0 g of chromium hydroxide green (CI77289) is added and well dispersed with a homogenizer to prepare an outer color coating solution.

A coating with the resulting outer color coating solution is proceeded by a fluidized bed process at the condition of 500 me/h of feeding rate of the coating solution to obtain particles having a titanium dioxide particle layer coated with an outer color layer.

Then, 200.0 g of polymethacrylate (Eudragit RSPO) is dissolved in 4000 g of ethanol to prepare an outermost protective layer coating solution. A coating with the resulting outmost shell coating solution is proceeded by a fluidized bed process at the condition of 100 ml/h of feeding rate of the coating solution to obtain a core-shell capsule having an outer color layer coated with a polymeric outermost shell.

### Example 6

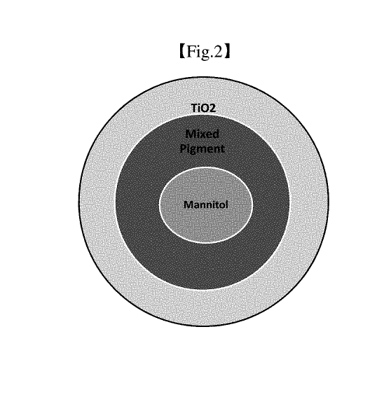

By using the ingredients and contents described in the below table 1, a color-changing microcapsule having 3 layers as shown in FIG. 2 is prepared by fluidized bed process:
(1) Mixed Pigment (Inner color layer): Yellow:Red:Black=55.18:34.48:10.34
(2) Layer Composition: Core seed-Inner color layer-TiO₂ particle layer

TABLE 1

| | | |
|---|---|---|
| (A) Core Seed | Mannitol | 16.45% |
| (B) Inner Color Layer | Mixed Pigment | 50.0% |
| | Lecithin | 0.4% |
| | Corn Starch binder | 2.0% |
| (C) TiO₂ Particle Layer | Titanium dioxide | 30.0% |
| | Lecithin | 0.2% |
| | Corn Starch binder | 0.8% |

### Example 7

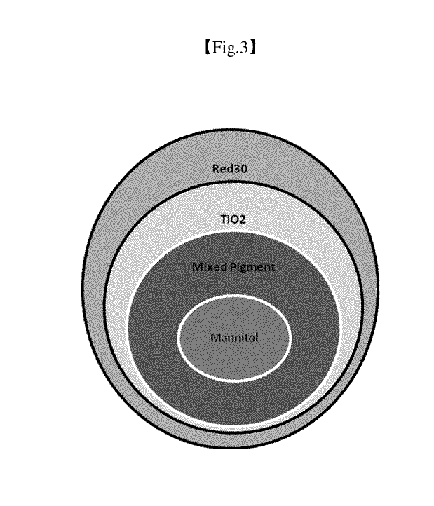

By using the ingredients and contents described in the below table 2, a color-changing microcapsule having 4 layers as shown in FIG. 3 is prepared by fluidized bed process:
(1) Mixed Pigment (Inner color layer): Yellow:Red:Black=60.4:23.8:11.4:4.4
(2) Layer Composition: Core seed-Inner color layer-TiO₂ particle layer-outer color layer

TABLE 2

| | | |
|---|---|---|
| (A) Core Seed | Mannitol | 6.5% |
| (B) Inner Color Layer | Mixed Pigment | 17.8% |
| | Sunpuro Yellow | 2.00% |
| | Lecithin | 5.0% |
| | Eudragit RSPO | 4.0% |
| (C) TiO₂ Particle Layer | Titanium dioxide | 55.0% |
| | Lecithin | 5.0% |
| | Eudragit RSPO | 4.0% |
| (D) Outer Color Layer | D&C Red30 | 0.8% |
| | Cornstarch binder | 0.4% |

### Example 8

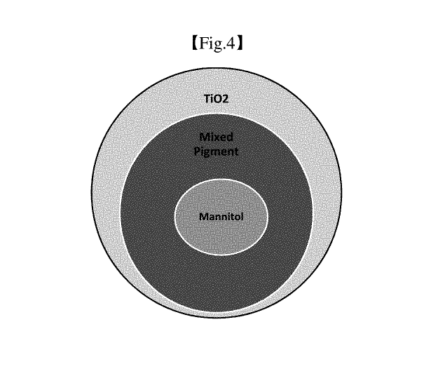

By using the ingredients and contents described in the below table 3, a color-changing microcapsule having 3 layers as shown in FIG. 4 is prepared by fluidized bed process:
(1) Mixed Pigment (Inner color layer): Yellow:Red:Black=60.1:28.8:11.1
(2) Layer Composition: Core seed-inner color layer-TiO₂ particle layer

TABLE 3

| | | |
|---|---|---|
| (A) Core Seed | Mannitol | 17.8% |
| (B) Inner Color Layer | Mixed Pigment | 19.8% |
| | Lecithin | 0.2% |
| | Corn Starch binder | 0.8% |
| (C) TiO₂ Particle Layer | Titanium dioxide | 50.0% |
| | Mannitol | 5.0% |
| | Corn Starch | 5.0% |
| | Lecithin | 0.3% |
| | Corn Starch binder | 1.2% |

### Example 9

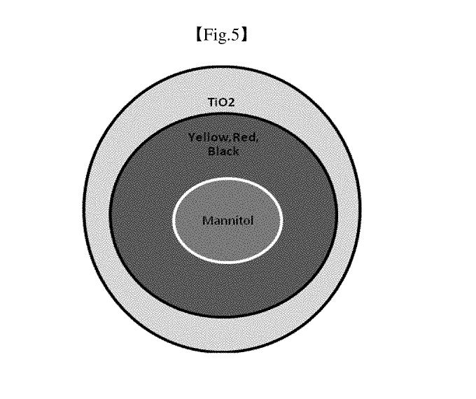

By using the ingredients and contents described in the below table 4, a color-changing microcapsule having 3 layers as shown in FIG. 5 is prepared by fluidized bed process,
(1) Mixed Pigment (Inner color layer): Yellow:Red:Black=80.2:17.0:2.8
(2) Layer Composition: Core seed-inner color layer-TiO₂ particle layer

TABLE 4

| | | |
|---|---|---|
| (A) Core Seed | Mannitol | 13.7% |
| (B) Inner Color Layer | Sunpuro Yellow | 17.36% |
| | Sunpuro Red | 3.67% |
| | Sunpuro Black | 0.61% |
| | Lecithin | 0.20% |
| | Corn Starch Binder | 1.0% |
| (C) TiO₂ Particle Layer | Titanium dioxide | 61.36% |
| | Lecithin | 0.3% |
| | Corn Starch Binder | 1.5% |

### Example 10

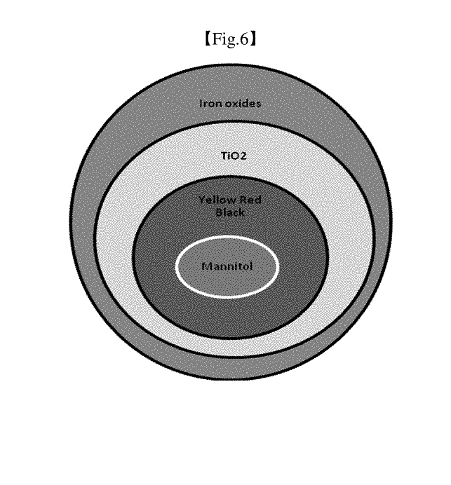

By using the ingredients and contents described in the below table 5, a color-changing microcapsule having 4 layers as shown in FIG. 6 is prepared by fluidized bed process,
(1) Mixed Pigment (Inner color layer): Yellow:Red:Black=55.18:34.48:10.34
(2) Layer Composition: Core seed-Inner color layer-TiO₂ particle layer-Outer color layer

TABLE 5

| | | | | |
|---|---|---|---|---|
| (A) Core-Seed | Mannitol | 16.81% | Mannitol | 16.81% |
| (B) Inner Color | Mixed | 49.15% | Iron oxide | 27.12% |
| Layer | Pigment | | Yellow | |
| | | | Iron oxide Red | 16.95% |
| | | | Iron oxide Black | 5.08% |
| | Lecithin | 0.29% | Hydrogenated | 0.29% |
| | | | Lecithin | |
| | CornStarch | 1.97% | Zea Mays(corn) | 1.97% |
| | Binder | | starch | |
| (C) TiO₂ Particle | Titanium | 29.49% | Titanium | 29.49% |
| Layer | dioxide | | dioxide | |
| | Lecithin | 0.1% | Hydrogenated | 0.1% |
| | | | Lecithin | |
| | Corn Starch | 0.49% | Zea Mays(corn) | 0.49% |
| | Binder | | starch | |
| (D) Outer Color | Sunpuro | 1.0% | Iron oxide | 1.0% |
| Layer | Yellow | | Yellow | |
| | Sunpuro Red | 0.2% | Iron oxide Red | 0.2% |
| | Corn Starch | 0.5% | Zea Mays(corn) | 0.5% |
| | Binder | | starch | |

### Example 11

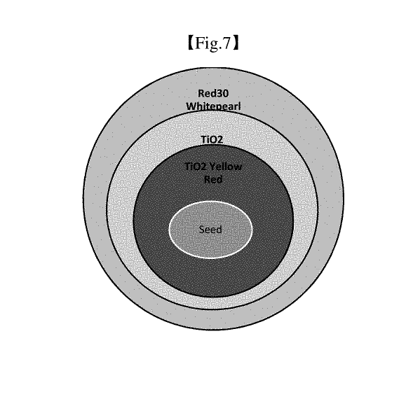

By using the ingredients and contents described in the below table 6, a color-changing microcapsule having 4 layers as shown in FIG. 7 is prepared by fluidized bed process,
(1) Mixed Pigment (Inner color layer): [symbol]
(2) Layer Composition: Core seed-Inner color layer-TiO₂ particle layer-Outer color layer

TABLE 6

| | | | | |
|---|---|---|---|---|
| (A) Core Seed | White4045 | 4.0% | Cellulose | 1.12% |
| | | | Mannitol | 1.0% |
| | | | Zea Mays(corn) | 1.84% |
| | | | starch | |
| | | | Hydrogenated | 0.04% |
| | | | Lecithin | |
| (B) Inner Color | Mixed | 55.0% | Titanium | 50.6% |
| Layer | Pigment | | Dioxide | |
| | | | Iron oxide | 3.3% |
| | | | Yellow | |
| | | | Iron oxide Red | 1.1% |
| | Lecithin | 0.50% | Hydrogenated | 0.50% |
| | | | Lecithin | |
| | Mannitol | 3.5% | Mannitol | 3.5% |
| | Corn Starch | 2.0% | Zea Mays(corn) | 2.0% |
| | Binder | | starch | |
| (C) TiO₂ Particle | Titanium | 5.0% | Titanium | 5.0% |
| Layer | dioxide | | dioxide | |
| | Corn Starch | 3.62% | Zea Mays(corn) | 3.62% |
| | | | starch | |
| | Cellulose | 9.0% | Cellulose | 9.0% |
| | Mannitol | 13.0% | Mannitol | 13.0% |
| | Lecithin | 0.25% | Hydrogenated | 0.25% |
| | | | Lecithin | |
| | Corn Starch | 1.8% | Zea Mays(corn) | 1.8% |
| | Binder | | starch | |
| (D) Outer Color | Satin White | 1.8% | Synthetic | 1.035% |
| Layer | | | Fluorphlogopite | |
| | | | Tin oxide | 0.009% |
| | | | Titanium | 0.756% |
| | | | Dioxide | |
| | D&C Red30 | 0.03% | Red30 Al. Lake | 0.03% |
| | Corn Starch | 0.5% | Zea Mays(corn) | 0.5% |
| | Binder | | starch | |

### Example 12

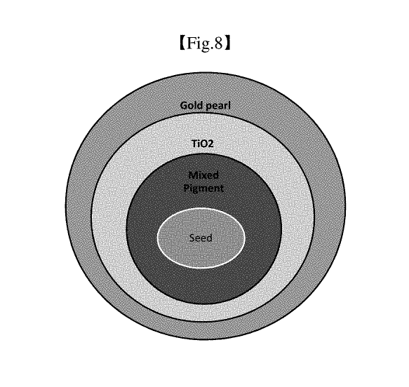

By using the ingredients and contents described in the below table 7, a color-changing microcapsule having 4 layers as shown in FIG. 8 is prepared by fluidized bed process,
(1) Mixed Pigment (Inner color layer): White:Yellow:Red:Black=89:2:8:1
(2) Layer Composition: Core seed-Inner color layer-TiO₂ particle layer-Outer color layer

TABLE 7

| | | | | |
|---|---|---|---|---|
| (A) Core-Seed | White2030 | 34.4% | Zea Mays(corn) | 14.3% |
| | | | Starch | |
| | | | Mannitol | 10.5% |
| | | | Cellulose | 9.6% |
| (B) Inner Color | Mixed | 50.0% | Titanium | 44.5% |
| Layer | Pigment | | Dioxide | |
| | | | Iron oxide | 4.0% |
| | | | Yellow | |
| | | | Iron oxide Red | 1.0% |
| | | | Iron oxide Black | 0.5% |
| | Lecithin | 0.50% | Hydrogenated | 0.50% |
| | | | Lecithin | |
| | Mannitol | 4.0% | Mannitol | 4.0% |
| | Corn Starch | 2.0% | Zea Mays(corn) | 2.0% |
| | Binder | | Starch | |
| (C) TiO₂ Particle | Titanium | 5.0% | Titanium | 5.0% |
| Layer | dioxide | | dioxide | |
| | Lecithin | 0.1% | Hydrogenated | 0.1% |
| | | | Lecithin | |
| | Corn Starch | 0.4% | Zea Mays(corn) | 0.4% |
| | Binder | | Starch | |
| (D) Outer Color | C. Monarch | 3.0% | Mica | 1.575% |
| Layer | gold | | Titanium | 1.29% |
| | | | Dioxide | |
| | | | Iron oxide Red | 0.12% |
| | | | Tin Oxide | 0.015% |
| | Corn Starch | 0.6% | Zea Mays(corn) | 0.6% |
| | Binder | | Starch | |

### Example 13

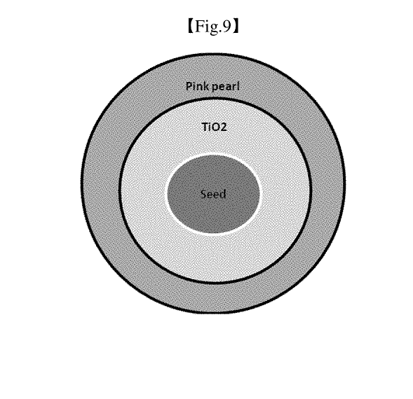

By using the ingredients and contents described in the below table 8, a color-changing microcapsule having 3 layers as shown in FIG. 9 is prepared by fluidized bed process,
(1) Layer Composition: Core seed-Inner color layer TiO₂ particle layer-Outer color layer

TABLE 8

| | | | | |
|---|---|---|---|---|
| (A) Core-Seed | Mannitol | 27.85% | Mannitol | 27.85% |
| (B) Inner Color | Titanium | 65.15% | Titanium | 65.15% |
| Layer (or (C) | dioxide | | dioxide | |
| | Lecithin | 0.5% | Lecithin | 0.5% |
| TiO₂ Particle | Corn Starch | 1.5% | Corn Starch | 1.5% |
| Layer) | Binder | | Binder | |
| (D) Outer Color | D&C Red30 | 0.145% | D&C Red30 | 0.145% |
| Layer | Satin White | 4.55% | Synthetic | 2.66% |
| | | | Fluorphlog- | |
| | | | opite | |
| | | | Tin oxide | 0.023% |
| | | | Titanium | 1.867% |
| | | | Dioxide | |
| | Corn Starch | 0.3% | Corn Starch | 0.3% |
| | Binder | | Binder | |

### Example 14

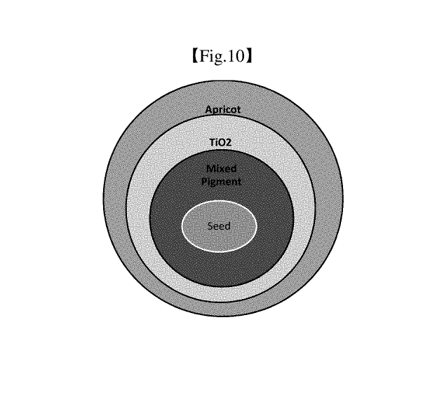

By using the ingredients and contents described in the below table 9, a color-changing microcapsule having 4 layers as shown in FIG. 10 is prepared by fluidized bed process:
(1) Mixed Pigment (Inner color layer): White:Yellow:Red:Black=84.3:5.0:8.7:2
(2) Layer Composition: Core seed-Inner color layer-TiO₂ particle layer-Outercolor layer

TABLE 9

| | | | | |
|---|---|---|---|---|
| (A) Core-Seed | White4045 | 4.0% | Cellulose | 1.0% |
| | | | Mannitol | 1.0% |
| | | | Zea Mays(corn) | 2.0% |
| | | | Starch | |
| (B) Inner Color | Mixed | 50.0% | Titanium | 42.15% |
| Layer | Pigment | | dioxide | |
| | | | Iron oxide | 2.5% |
| | | | Yellow | |
| | | | Iron oxide Red | 4.35% |
| | | | Iron oxide Black | 1.0% |
| | Lecithin | 0.50% | Hydrogenated | 0.50% |
| | | | Lecithin | |
| | Mannitol | 3.5% | Mannitol | 3.5% |
| | Corn Starch | 2.0% | Zea Mays(corn) | 2.0% |
| | Binder | | Starch | |
| (C) TiO₂ Particle | Titanium | 5.0% | Titanium | 5.0% |
| Layer | dioxide | | dioxide | |
| | Corn Starch | 2.0% | Zea Mays(corn) | 2.0% |
| | | | Starch | |
| | Cellulose | 5.0% | Cellulose | 5.0% |
| | Mannitol | 6.5% | Mannitol | 6.5% |
| | Lecithin | 0.25% | Hydrogenated | 0.25% |
| | | | Lecithin | |
| | Corn Starch | 1.0% | Zea Mays(corn) | 1.0% |
| | Binder | | Starch | |
| (D) Outer Color | Iron oxide Red | 0.05% | Iron oxide Red | 0.05% |
| Layer | Iron oxide | 0.01% | Iron oxide | 0.01% |
| | Yellow | | Yellow | |
| | Cellulose | 5.0% | Cellulose | 5.0% |
| | Mannitol | 6.5% | Mannitol | 6.5% |
| | Corn Starch | 7.44% | Zea Mays(corn) | 7.44% |
| | | | Starch | |
| | Lecithin | 0.25% | Hydrogenated | 0.25% |
| | | | Lecithin | |
| | Corn Starch | 1.0% | Zea Mays(corn) | 1.0% |
| | Binder | | Starch | |

### INDUSTRIAL APPLICABILITY

The color-changing microcapsule according to the present invention have a high durability during storage and handling and a high masking ability of inner color as well as can maintain a long period stability.

1-18. (canceled)

19. A color-changing microcapsule having an average diameter of 50 μm-1500 μm and having a core shell structure, wherein said core comprises the following core seed (A) and at least one inner color layer (B) and said shell is pressure breakable wall layer (C):
(A) core seeds having an average diameter of 500 nm-150 μm, no colorant, and containing sugar alcohols;
(B) at least one inner color layer comprising:
more than one colorant; and
a binder comprising at least one wall forming material and at least one lipid based material; and
(C) a pressure breakable wall layer selected from 10 μm-500 μm in thickness and comprising:
titanium dioxide particles, and
a binder comprising at least one wall forming material and at least one lipid based material.

20. The color-changing microcapsule of claim 19, wherein said core comprises two or three of the following inner color layers, selected from the group consisting of:
(B-1) a first inner color layer comprising:
at least one colorant, and
a binder comprising at least one wall forming material and at least one lipid based material; and
(B-2) a second inner color layer comprising:
at least one colorant, and
a binder comprising at least one wall forming material and at least one lipid based material; and
(B-3) a third inner color layer comprising:
at least one colorant, and
a binder comprising at least one wall forming material and at least one lipid based material;
wherein, the above-mentioned colorants, wall-forming materials and lipid-based materials used in (B-1), (B-2) and (B-3) are the same or different from each other.

21. The color-changing microcapsule according to claim 19, wherein said shell comprises one or both of the following outer color layer (D) and outermost protective layer (E):
(D) at least one outer color layer surrounding the pressure breakable wall layer and comprising:
at least one colorant, and
a binder comprising at least one wall forming material and at least one lipid based material; and
(E) An outermost protective layer surrounding the pressure breakable wall layer or outer color layer and comprising:
a shell forming polymer selected from the group consisting of shell rock, polyacrylate, polymethacrylate, cellulose ether, cellulose ester, polystyrene maleic anhydride copolymer, and mixtures thereof.

22. The color-changing microcapsule according to claim 19, wherein the sugar alcohol is selected from the group consisting of erythritol, traitol, arabitol, xylitol, ribitol, mannitol, sorbitol, galactitol, iditol, inositol, volemitol, and mixtures thereof:

23. The color-changing microcapsule according to claim 19, wherein the pressure breakable wall layer (C) comprises:
5-99% by weight of titanium dioxide particles;
0.1-30% by weight of at least one wall forming material;
0.1-30% by weight of at least one lipid based material.

24. The color-changing microcapsule according to claim 19, wherein the thickness ratio between the core-seed and the inner color layer is selected from 0.1:1-1:0.1.

25. The color-changing microcapsule according to claim 19, wherein it has a mean particle size of 100 μm-800 μm.

26. The color-changing microcapsule according to claim 19, wherein said wall-forming material is a hydrophilic polymer capable of forming a hydrogen bond with water or an alcohol compound.

27. The color-changing microcapsule according to claim 19, wherein the lipid-based material is selected from sphingolipids or phospholipids.

28. The color-changing microcapsule of claim 27, wherein the lipid-based material is selected from ceramides, lecithin, or hydrogenated lecithin.

29. The color-changing microcapsule according to claim 19, wherein said colorant is an inorganic pigment or a glass pigment.

30. The color-changing according to claim 29, wherein said colorant is at least one colorant selected from the group consisting of yellow iron oxide, red iron oxide, black iron oxide, chromium oxide green, chromium hydroxide green and ultramarine blue Microcapsules.

31. The color-changing microcapsule according to claim 29, wherein the colorant of the inner color layer is titanium dioxide.

32. A process for producing color-changing microcapsules according to claim 19, comprising the steps of:
(a) preparing core-seed (A) particles;
(b) coating the core-seed (A) particles with a solution in which a colorant and a binder are dispersed or dissolved to form an inner color layer (B); and
(c) coating the particles obtained in the step (b) with a solution in which titanium dioxide particles and a binder are dispersed or dissolved to form a pressure breakable wall layer (C),
wherein the binder described above comprises a wall forming material and a lipid-based material, wherein the wall forming material and the lipid based material described above are the same or different from each other.

33. The method according to claim 16, wherein the step (b) comprises the following steps (b-1) and (b-2):
(b-1) coating the core-seed (A) particles with a solution in which a colorant and a binder are dispersed or dissolved to form a first inner color layer (B-1); and
(b-2) coating the particles obtained in the step (b-1) with a solution in which a colorant and a binder which are the same as or different from those used in the step (b-1) are dispersed or dissolved to form a second internal growth layer (B-2),
wherein the binder described above comprises a wall forming material and a lipid based material, wherein the wall forming material and the lipid based material described above are the same or different from each other.

34. The method of claim 32, further comprising one or both of the following steps (d) and (e):
(d) The particles obtained in the step (c) are coated with a solution in which a colorant and a binder which are the same or different as those used in the step (b-1) or (b-2) are dispersed or dissolved to form an external color layer (D),
(e) The particles obtained in the step (c) or (d) are coated with a solution in which a shell-forming polymer is dispersed or dissolved to form an outermost protecting layer (E);
wherein, the binder described above comprises a wall-forming material and a lipid-based material, wherein the wall-forming material and the lipid-based material described above are the same or different from each other.

35. The method of claim 32, wherein said coatings in steps (b), (b-1), (b-2), (c), (d) and (e) are proceed by the fluidized bed method.

36. The method of claim 32, wherein said solution comprises a solvent selected from the group consisting of methylene chloride, methanol, and ethanol.
